(19) United States
(12) Patent Application Publication
(10) Pub. No.: US 2003/0120114 A1
(43) Pub. Date: Jun. 26, 2003

(54) Preparation of fluorophenylhydrazines and salts thereof

(76) Inventor: Friedrich-Wilhelm Ullrich, Bergisch Glabdach (DE)

Correspondence Address: BAYER CHEMICALS CORPORATION, 100 BAYER ROAD, PITTSBURGH, PA, 15205, US

(21) Appl. No.: 10/314,808

(22) Filed: Dec. 9, 2002

(30) Foreign Application Priority Data
Dec. 12, 2001 (DE) 10161198.6

(51) Int. Cl. C07C 243/22

(52) U.S. Cl. 564/310

(57) ABSTRACT

A process is provided for preparing fluorophenylhydrazines and salts thereof in high yields by a simple route of diazotizing fluoroanilines, reducing, hydrolysing and optionally neutralizing.

### BACKGROUND OF THE INVENTION

1. Field of the Invention

The invention relates to a process for preparing fluorophenylhydrazines and salts thereof by diazotizing fluoroanilines, reducing, hydrolysing and optionally neutralizing.

2. Brief Description of the Prior Art

Fluorophenylhydrazines having the basic structure of the following formula

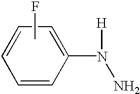

are valuable intermediates for preparing active ingredients in the agrochemical (U.S. Pat. No. 5,449,784) and the pharmaceutical industries (DE-A-198 16 882).

Substituted phenylhydrazines are prepared by diazotizing the corresponding aniline and then reducing the diazonium salt. Reducing agents used in the literature include a large number of compounds, for example zinc and tin(II) chloride. However, the reducing agent of industrial significance is in particular the system composed of sodium sulphite, sodium bisulphite and sulphuric acid.

Illustratively, EP-A-0 723 953 describes a process for preparing 2-fluorophenylhydrazine or 2 fluorophenylhydrazine hydrochloride. In the process, 2-fluoroaniline is diazotized, and the diazonium salt obtained is reduced using bisulphite liquor to the 2 fluorophenylhydrazine-α,β-disulphonate; the latter is hydrolysed using hydrochloric acid to give 2-fluorophenylhydrazine hydrochloride; alkali metal hydroxide is added to neutralize; then the mixture is cooled, and the precipitate of 2-fluorophenyl-hydrazine is filtered off and optionally reacted with hydrochloric acid to give 2-fluorophenylhydrazine hydrochloride. While it is essential to use a defined excess of hydrochloric acid in the diazotization step, in the range of 3-3.5, in particular 3.2, moles of hydrochloric per mole of 2-fluoroaniline, the associated large excess of mineral acid leads to a large acid burden on the resulting waste water and to corrosion problems in the reactor tanks used. The high proportion of mineral acid in the waste water leads to an increase in the waste water disposal costs and to increased technical cost and inconvenience for regenerating the mineral acid. EP-A-0 723 953 further describes particular preference for carrying out the reduction of the diazonium salt at a pH in the range from 4.5 to 7.5. The maintenance of this pH is decisive for the ratio of sodium sulphite, sodium bisulphite and sulphuric acid in the system. However, since the first diazotization step requires a large excess of hydrochloric acid, the subsequent reduction step requires firstly a considerable initial charge of sodium hydroxide solution, and further metering in of sodium hydroxide solution at the same time as the diazonium salt is added to the sulphite mixture. The yield obtained by this process is 91% of theory.

U.S. Pat. No. 6,087,534 likewise discloses the preparation of arylhydrazines by contacting the appropriate diazonium compound with a bisulphite liquor having a pH of at least 7 and preferably 7.5 to 10. A disadvantage of this process is that nitrogen bases, especially ammonia, are used to adjust the pH. These nitrogen bases are inevitably added to the waste water in the form of ammonium salts and have to be removed again by costly and inconvenient measures. This process provides the arylhydrazines in a yield of 92% of theory. EP-A-0 959 067 describes the preparation of arylhydrazines by hydrolysing aryl-hydrazine α,β-disulphonates in the presence of an inert organic solvent using water and an inorganic acid and subsequent reaction of the resulting arylhydrazine salt with a base to give arylhydrazine. In many cases, the presence of the solvent in the hydrolysis allows the quantity of inorganic acid to be used in the hydrolysis step to be reduced, in particular in the case of unstable arylhydrazine salts. Little information is given on the preceding diazotization and reduction. In the preceding diazotization, the molar ratios of 2-fluoroaniline to hydrochloric acid in the examples of 1:2.5 and 1:2.4 are distinctly lower than in the process of EP-A-0 723 953. However, this process provides only unsatisfactory yields of 74-78% of theory.

The object of the present invention is accordingly to provide a process by which fluorophenylhydrazines are obtained in improved yields in a technically simple manner.

### SUMMARY OF THE INVENTION

The invention provides a process for preparing fluorophenylhydrazines or salts thereof by

1) diazotizing fluoroanilines in the presence of hydrochloric acid using a molar ratio of fluoroaniline to hydrochloric acid of 1:(92 to 2.9),

2) reducing in the presence of bisulphite liquor with the addition of alkali metal hydroxide solution at a pH in the range from 5 to 8 to form the fluorophenylhydrazine α,β-disulphonate,

3) hydrolysing the fluorophenylhydrazine α,β-disulphonate using mineral acids in an aqueous medium which is free of organic solvents and

4) optionally adding a base to neutralize.

Useful fluoroanilines in the process according to the invention include those of the formula (I)

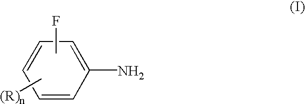

where R is a straight-chain or branched C₁-C₄-alkyl radical and n is 0, 1, 2, 3 or 4.

The fluorine radical may be in the 2-, 3- or 4-position to the NH₂ radical in formula (I), but is preferably in the 2-position. R is preferably methyl or ethyl and n is preferably 1. Preference is also given to n being 0. Particular preference is giving to using 2-fluoroaniline.

For the diazotization in step 1) of the process according to the invention, hydrochloric acid and fluoroaniline are used in a molar ratio of (2-2.9):1. Preference is given to using a molar ratio of (2.2-2.6):1.

The diazotization is customarily carried out with the addition of sodium nitrite as the diazotizing reagent. The diazotizing reagent is metered in with cooling until a positive nitrite spot test is obtained using iodine-starch paper.

The reduction in step 2) of the process according to the invention of the diazonium salt obtained in step 1) is carried out with the use of a bisulphite liquor. This bisulphite liquor is an aqueous solution of sodium sulphite, sodium bisulphite and sulphuric acid. The ratio of sodium sulphite, sodium bisulphite and sulphuric acid in the reaction mixture is determined by the pH of the reaction mixture. Using sodium hydroxide solution, the pH is adjusted to 5 to 8, preferably 6 to 7. The molar ratio of the sum total of sodium sulphite, sodium bisulphite and sulphuric acid to the fluoroaniline used is in the range of 2-3, preferably in the range of 2-2.4.

It has proven useful to meter the diazonium salt solution into the initially charged bisulphite liquor whilst customarily maintaining a temperature in the range of 30-100° C. and preferably in the range of 50-80° C. in the reaction mixture. This reduction forms the fluorophenylhydrazine α,β-disulphonate from the diazonium salt.

In the third step, this fluorophenylhydrazine α,β-disulphonate is hydrolysed by contacting with a mineral acid in an aqueous reaction medium which is free of organic solvent. Even catalytic quantities of the mineral acid are sufficient, and higher quantities accelerate the reaction. It is advantageous to meter the fluorophenylhydrazine α,β-disulphonate into the mineral acid, since this method allows sulphuric acid released from the excess bisulphite to be constantly removed. Preference is given to using hydrochloric acid as the mineral acid.

The solution or suspension of the desired 2-fluorophenylhydrazinium salt generated in step 3) may optionally be introduced directly to a subsequent reaction without isolation. However, it is also possible to isolate the salt by common prior art methods, for example crystallization out of the reaction mixture.

When fluorophenylhydrazine itself is to be isolated, neutralization is effected in step 4) as follows: the reaction mixture is adjusted using a suitable base, preferably an alkali metal hydroxide solution, in particular sodium hydroxide solution, to a pH>8, preferably to a pH of 9-12, more preferably to a pH of 10-11. The free hydrazine is then extracted using a suitable organic solvent. Examples of organic solvents which have proven useful include aliphatic ethers or aromatic compounds, for example toluene, xylene or chlorobenzene. It will be appreciated that the possibility also exists at this point of using fluorophenylhydrazine in the form of the solution generated in this manner in the subsequent chemistry, or of concentrating the solution to obtain the neat fluorophenylhydrazine.

Compared to the free hydrazines, the salts thereof have a higher storage stability. They may in turn be obtained from a solution of the free hydrazines by adding an organic acid such as acetic acid, or a mineral acid such as hydrochloric or sulphuric acid. Preference is given to converting fluorophenylhydrazines to the hydrochloride. This may be generated from the solution of the free hydrazine by adding hydrochloric acid dropwise or by passing in hydrogen chloride gas. Fluorophenylhydrazine hydrochloride is isolated as a solid by filtering off with suction and drying.

Surprisingly, the process according to the invention may be carried out using a smaller molar excess of hydrochloric acid to 2-fluoroaniline than the process of EP 0 723 953. This was not to be expected, since EP-A-0 723 953 refers several times to the necessity of maintaining the abovementioned high molar ratio of hydrochloric acid to fluoroaniline. According to EP-A-0 959 067, the use of a smaller molar ratio of HCl:2-fluoroaniline of 2.4 or 2.5:1 gives distinctly worse results. The lower molar ratio in the process according to the invention brings several advantages: as well as a simple saving of hydrochloric acid, there is a corresponding equivalent saving of sodium hydroxide solution in the reduction step and the resulting reduction of waste water volumes and salt burden. This is accompanied by a corresponding increase in space-time yield. A further advantageous effect is observed when metering the diazonium salt solution into the bisulphite mixture: once the pH has been set beforehand, it remains during metering and further stirring time within a narrow optical range without additional measures, such as the simultaneous metering in of sodium hydroxide solution as disclosed by EP-A-0 723 053, being necessary. Another advantage of the process according to the invention is that the organic solvents which are essential in EP-A-0 959 067 are not required in the hydrolysis step. As well as these advantages, the process according to the invention also leads to a higher product yield.

### EXAMPLES

### Example 1

Diazotization

55.6 g of 2-fluoroaniline (0.5 mol) are added dropwise with vigorous mixing to 100 ml of water and 127 ml of 30% hydrochloric acid (1.2 mol) at room temperature. The resulting suspension of 2-fluoroaniline hydrochloride is admixed at 0-5° C. with 120 ml of a 20% sodium nitrite solution (0.52 mol).

Sulphite Reduction

The 2-fluorophenyldiazonium chloride solution obtained from the diazotization is metered within 40 minutes into a solution of 215 ml of bisulphite liquor (39%, 1.08 mol) heated to 60° C. which had previously been set to pH 6.5 using 45 ml of sodium hydroxide solution. The batch is stirred for an hour at 60° C., and the pH at the end of this time is 6.3.

Hydrolysis

A further reactor is charged with 50 ml of hydrochloric acid (30%) and heated to 100° C. Within 30 minutes, the sulphite reduction batch is metered into the hydrochloric acid. The batch is stirred at 100° C. for 2 hours, and is then cooled to 60° C.

Isolation

80 ml of sodium hydroxide solution (45%) and 100 ml of toluene were initially charged. The 2-fluorophenylhydrazinium salt solution was added dropwise. At the end of the metering in, the pH was 10 to 11. At 40° C., the phases were separated. The aqueous phase was washed twice more with 100 ml of toluene each time. The combined organic phases were admixed at room temperature with 62 ml of hydrochloric acid (37%). 2-fluorophenylhydrazine hydrochloride precipitated out and was filtered off with suction and dried. 78.1 g were obtained having a purity of 99%. This corresponds to a yield of 95% of theory.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for preparing fluorophenylhydrazines or salts thereof comprising the steps of:
1) diazotizing fluoroanilines in the presence of hydrochloric acid using a molar ratio of fluoroaniline to hydrochloric acid of 1:(2 to 2.9),
2) reducing in the presence of bisulphite liquor by adding alkali metal hydroxide solution at a pH in the range from 5 to 8 to form 2-fluorophenylhydrazine α,β-disulphonate,
3) hydrolysing the 2-fluorophenylhydrazine α,β-disulphonate using mineral acids in an aqueous medium which is free of organic solvents and
4) optionally adding alkali metal hydroxide solution to neutralize.

2. Process according to claim 1, wherein step 1 is carried out using fluoroanilines of the formula (I) 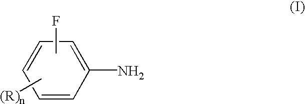
where R is a straight-chain or branched C1-C4-alkyl radical and n is 0, 1, 2, 3 or 4.

3. Process according to claim 2, wherein the fluorine radical is in the 2-position to the NH2 radical in formula (I).

4. Process according to claim 2, wherein R is methyl or ethyl and n is 1.

5. Process according to claim 1, wherein hydrochloric acid and fluoroaniline are used in a molar ratio of (2.2 to 2.6):1.

6. Process according to claim 1, wherein the diazotization is carried out with the addition of sodium nitrite as the diazotizing reagent.

7. Process according to claim 1, wherein the reduction is carried out at a pH of 6 to 7.

8. Process according to claim 1, wherein the diazonium salt in solution is metered into the bisulphite liquor initially charged, and the resulting reaction mixture is maintained within a temperature range of 30 to 100° C.

9. Process according to claim 1, wherein the hydrolysis is carried out with the addition of hydrochloric acid.

10. Process according to claim 1, wherein fluorophenylhydrazine is isolated by adjusting the pH in step 4) using an alkali metal hydroxide solution, and extracting the fluorophenylhydrazine using a suitable organic solvent.

11. Process according to claim 10, wherein fluorophenylhydrazine salts are isolated after step 4) by admixing the solution of the fluorophenylhydrazine in the organic solvent with an organic acid or a mineral acid, and isolating the fluorophenylhydrazine salt formed.
